(19) United States
(12) Patent Application Publication
(10) Pub. No.: US 2018/0162391 A1
(43) Pub. Date: Jun. 14, 2018

(54) VEHICLE CONTROL METHOD AND VEHICLE CONTROL APPARATUS FOR PREVENTING RETALIATORY DRIVING

(71) Applicant: INFOBANK CORP., Seongnam-si (KR)

(72) Inventors: Ji Sung KIM, Seoul (KR); Sang Gyeong BAE, Gwangmyeong-si (KR); Kwang Rim HA, Seoul (KR); Chang Seok YOUK, Bucheon-si (KR)

(73) Assignee: Infobank Corp.

(21) Appl. No.: 15/646,947

(22) Filed: Jul. 11, 2017

(30) Foreign Application Priority Data
Dec. 8, 2016 (KR) 10-2016-0166801

(51) Int. Cl. B60W 30/09; B60W 30/095; B60W 40/04; B60W 40/09; B60W 40/107; B60W 50/10; B60W 50/12; G08G 1/16; G08G 1/04; B60K 35/00; A61B 5/024; A61B 5/18

(52) U.S. Cl. CPC B60W 30/09; B60K 2350/962; B60W 40/04; B60W 40/09; B60W 40/107; B60W 50/10; B60W 50/12; G08G 1/166; G08G 1/04; B60K 35/00; A61B 5/02438; A61B 5/18; B60W 2420/42; B60W 2520/105; B60W 2720/106; B60W 2750/40; B60W 2550/22; B60W 2550/30; B60W 30/095

(57) ABSTRACT

A vehicle control method according to at least one embodiment of the present disclosure includes collecting vehicle driving information and/or sensor information, identifying a traveling pattern of a driver from the vehicle driving information and/or the sensor information, determining information on a retaliatory driving corresponding to the driver based on the traveling pattern, and executing a processing corresponding to the information on the retaliatory driving.

### BACKGROUND OF THE INVENTION

### 1. Field of the Invention

The present disclosure relates to a vehicle control method and a vehicle control apparatus for preventing retaliatory driving.

### 2. Description of the Related Art

With the rapid increase of vehicle use and the proportional number of traffic accidents on the road, vehicle accidents involve human casualties as well as physical damages such as vehicle breakdown.

Intentional or not, there are cases where vehicle drivers can be in danger of accident due to overtaking or decelerating operation of other vehicles.

Drivers in that situation may be psychologically threatened at the least, and some aggressively react with reckless driving while also committing road rage actions as retaliation to other vehicles that have put their vehicles in danger.

In this way, the act of reckless driving for retaliation is increasing day by day. Recently, various media, SNS, etc. report increased risk of the ferocity of retaliatory driving by road ragers neglecting even the most basic social rules.

Some aggressive driving actions include honking horns while chasing other cars, threatening with the high beam, violating the centerline, blocking the traffic, spitting threats and abuse at other cars. Some are escalated to intentionally cutting off other drivers or braking suddenly, and going as far as pushing the running vehicle through the centerline and the shoulder, which have become more frequent in our society.

Such retaliatory driving is an imminent major accident to harm a lot of people, and therefore the committer is supposed to strictly take responsibility. There is a dire need for a preventive measure because many actual cases have occurred on a freeway where retaliatory driving incidents ended up road casualties.

However, in current vehicle systems, there is no preventive measure or apparatus associated with retaliatory driving at all. Systems are in existence for protecting the driver, but they are incapable of preventing retaliatory driving actions. For example, a dangerous driving event determined only by the running speed hardly confirms if a classified retaliatory driving is committed or not.

In other words, even if the driver of the current vehicle travels with the intention of retaliatory driving or subject to retaliation from another vehicle, the conventional system neither can identify the retaliatory intention easily nor can barely inform the involved drivers of the situation to control in advance.

### SUMMARY OF THE INVENTION

The present disclosure in some embodiments seeks to resolve the above-mentioned issues, and provides a vehicle control method and a vehicle control apparatus for confirming a retaliatory driving occurred and physically controlling the operation of the vehicle to thereby prevent the retaliatory driving from developing, or providing the user with retaliatory driving-related information to take a preventive or defensive action against the retaliatory driving and supply the information on the incident.

According to at least one embodiment of the present disclosure to solve the above-mentioned issues, there is provided a method of controlling a vehicle, including collecting vehicle driving information or driver's sensor information, identifying a traveling pattern of the driver from the vehicle driving information or the sensor information, determining information on a retaliatory driving corresponding to the driver based on the traveling pattern, and executing a processing corresponding to the information on the retaliatory driving.

An apparatus for controlling a vehicle according to an aspect of the present invention, includes a driving information collecting unit configured to collect vehicle driving information or sensor information; a traveling pattern processing unit configured to identify a traveling pattern of a driver from the vehicle driving information and/or the sensor information; and a control unit configured to determine information on a retaliatory driving corresponding to the driver based on the traveling pattern, and to execute a processing corresponding to the information on the retaliatory driving.

The method according to the present invention may be implemented as a program for execution on a computer and stored in a computer-readable recording medium.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a conceptual diagram schematically showing an overall system according to an embodiment of the present

FIG. 2 is a block diagram illustrating a vehicle control apparatus according to an embodiment of the present invention in more detail.

FIG. 3 is a flowchart illustrating an operation of the vehicle control apparatus according to the embodiment of the present invention in more detail.

FIG. 4 is a flowchart illustrating an operation of the vehicle control apparatus according to another embodiment of the present invention.

FIGS. 5 to 7 are views showing interface screens of the vehicle control apparatus according to the embodiment of the present invention.

### DETAILED DESCRIPTION OF THE EMBODIMENTS

While the invention is susceptible to various modifications and alternative forms, specific embodiments thereof are shown by way of example in the drawings and will herein be described in detail.

It is to be understood that the present invention is not intended to be limited to the specific embodiments but includes all changes, equivalents, and alternatives falling within the spirit and scope of the present invention. In describing the present invention, the terms first, second, etc. may be used to describe various elements, but the elements may not be limited by the terms. The terms may only be used for the purpose of distinguishing one element from another. For example, without departing from the scope of the present invention, the first component may be referred to as a second component, and similarly, the second component may also be referred to as a first component.

Where an element is referred to as being “coupled” or “connected” to another element, it may be directly coupled or connected to the other element, but it can be understood that other elements may be present in between. On the other hand, when an element is referred to as being “directly coupled” or “directly connected” to another element, it can be understood that there are no other elements in between. The terminology used herein is for the purpose of describing particular embodiments only and is not intended to limit the invention. The singular expressions may include plural expressions unless the context clearly dictates otherwise. In this specification, the terms “comprise”, “having”, and the like are used interchangeably to designate the presence of stated features, numbers, steps, operations, configurations, parts or combinations thereof, but do not preclude the presence or possibility of the addition of one or more other features, numbers, steps, operations, elements, parts, or combinations thereof. Unless defined otherwise, all terms used herein, including technical or scientific terms, may have the same meaning as commonly understood by one of ordinary skill in the art to which this invention belongs.

Terms such as those defined in commonly used dictionaries may be interpreted as having a meaning consistent with the meaning in the context of the related art and unless explicitly defined in the present application, may not be interpreted in an ideal or overly formal sense. In addition, the following embodiments are provided to more fully explain the present invention to those skilled in the art, and the shapes and sizes of elements in the drawings may be exaggerated for a clearer description.

Also, for example, block diagrams herein should be understood to represent conceptual aspects of exemplary circuits embodying the principles of the invention. The functions of the various elements shown in the drawings, including the functional blocks shown as a processor or similar concept, may be provided by use of dedicated hardware as well as hardware capable of executing software in connection with appropriate software. When provided by a processor, the functions may be provided by a single dedicated processor, a single shared processor, or a plurality of individual processors, some of which may be shared. Also, explicit use of terms such as processor, control, or similar concepts should not be interpreted exclusively as hardware capable of running software, and may include, without limitation, digital signal processor (DSP) hardware, read only memory (ROM), random access memory (RAM), and non-volatile memory for storing software. Other well-known hardware may also be included.

In the claims of the present specification, components represented as means for performing the functions described in the detailed description include all methods of performing functions comprising all types of software, including, for example, a combination of circuit elements performing the function or firmware/microcode, etc., and are coupled with appropriate circuitry for executing the software to perform the functions. As the functions provided by the various listed means are combined and they are combined with the manner in which the claims require, it is to be understood that the invention as defined by the appended claims is to be construed as encompassing any means capable of providing such functionality, which are equivalent to those understood from the present specification.

Hereinafter, a preferred embodiment of the present invention will be described in detail with reference to the accompanying drawings.

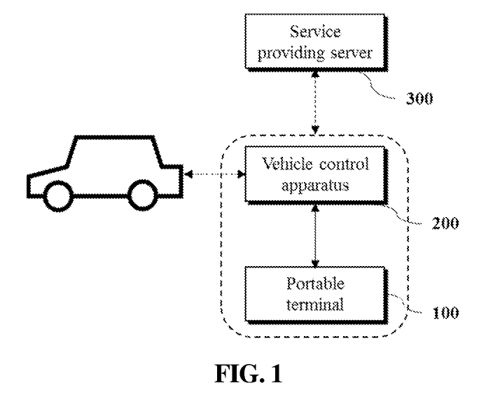

FIG. 1 is a conceptual diagram schematically illustrating an overall system according to an embodiment of the present invention.

Referring to FIG. 1, an overall system according to an embodiment of the present invention includes a portable terminal 100, a vehicle control apparatus 200, and a service providing server 300. In the embodiment of the present invention, the vehicle control apparatus 200 may be implemented as a system for controlling and managing a vehicle, and may be interlocked with the portable terminal 100. The vehicle control apparatus 200 can control one or more electric functions provided in the vehicle on its own or in accordance with the communication with the portable terminal 100.

For example, the portable terminal 100 can be interlocked with the vehicle control apparatus to configure an integrated system by providing a variety of information that can provide, complement, and augment the function of a navigation system of a vehicle. The terminal 100 in the entire system can be implemented as various devices provided or mounted in the vehicle.

For example, the portable terminal 100 may be implemented as a conventional vehicle information providing terminal provided in a vehicle (for example, an Audio Video Navigation (AVN) system implemented as a single system in which audio and multimedia devices, a navigation device, and the like are integrated). Further, the terminal 100 may be implemented as various devices such as, not only an AVN system, but also a navigation system capable of performing route guidance to a specific point, a black box, smart phone, tablet computer, personal digital assistant (PDA), a portable multimedia player (PMP), or the like for photographing the vicinity of the vehicle and storing or transmitting image information, and may also be implemented in a form capable of connecting to a mobile communication network.

In addition, the service providing server 300 may provide data which serves as a basis of the retaliatory driving related information provided through the vehicle control apparatus 200 interlocked to the portable terminal 100, or is capable of storing and managing retaliatory driving related information received from the vehicle control apparatus.

Accordingly, the vehicle control apparatus 200 can be connected to the terminal 100 either wirelessly or by wire and can be provided inside the vehicle, and the user can control the portable terminal 100 through the vehicle control apparatus 200, and may control the vehicle control apparatus 200 by using the portable terminal 100.

The entire system network including the terminal 100, the vehicle control apparatus 200, and the service providing server 300 according to an embodiment of the present invention may be connected by a wired or wireless network. In case of a wired one, it can be connected with HDMI, VGA, sRGB, USB interface or other cable interfaces. In case of a wireless one, it can be connected through Bluetooth, RFID (Radio Frequency Identification), Infrared Data Association (IrDA), Ultra WideB and (UWB), Zigbee, and so on. In addition, in the case of the portable terminal 100, upon receiving a call signal through a base station 10 and a mobile communication network 20, an operation of a basic function may be processed.

Meanwhile, the vehicle control apparatus 200 includes one or more control systems connected to each electric component of the vehicle to perform control for operating the vehicle. Generally, an electric control unit (ECU) can be taken as an example.

Also, the vehicle control apparatus 200 can control the operation of the vehicle in accordance with the user vehicle operation input.

Particularly, in the embodiment of the present invention, the vehicle control apparatus 200 collects vehicle driving information or sensor information of a driver, identifies a driving pattern of the driver from the vehicle driving information or the sensor information, determines retaliatory driving related information corresponding to the driver based on the driving pattern, and performs a process corresponding to the retaliative driving related information, thereby an appropriate process for preventing retaliatory driving may be performed.

More specifically, the vehicle control apparatus 200 combines the driving information of the vehicle and the sensor information for the driver who is driving the vehicle, identifies the driving pattern of the driver based thereon, and can determine whether or not the driving pattern corresponds to the retaliatory driving pattern.

To this end, the vehicle control apparatus 200 may collect all or a part of the vehicle driving information or the sensor information of the driver from the electric elements provided inside the vehicle.

Further, the vehicle control apparatus 200 may collect all or a part of the vehicle driving information or the sensor information of the driver from the portable terminal 100 interlocked with the vehicle control apparatus 200.

In addition, the vehicle control apparatus 200 may use retaliatory driving pattern information stored in advance or provided from the service providing server 300 or the portable terminal 100 so that it can be determined whether or not the vehicle driving information and the sensor information of the driver corresponds to the retaliatory driving pattern.

Accordingly, the vehicle control apparatus 200 can perform an appropriate process according to the retaliatory driving pattern. To this end, the vehicle control apparatus 200 may output a warning message to the user, output punishment regulation information, or perform a restriction process of the vehicle operation through the portable terminal 100 or a self warning electric element.

In particular, in order to limit the vehicle operation, the vehicle control apparatus 200 can identify the type according to the pattern of the retaliatory driving and may process the corresponding acceleration or speed limitation.

On the other hand, with the interlocking of the portable terminal 100 or the service providing server 300, and based on the vehicle driving information and sensor information of the driver, the control apparatus 200 can identify the case where it is subject to retaliatory driving by another vehicle nearby.

To this end, the vehicle control apparatus 200 can identify the retaliatory driving pattern of another vehicle due to the nearby vehicle based on not only the driver but also the black box and the sensor information, and can perform a process accordingly.

Here, the above process can be exemplified by various processes. In the case where the retaliatory driving pattern is identified, the vehicle control apparatus 200 outputs punishment regulation information corresponding thereto or provides a call connection to a corresponding service providing server 300, or may perform a process of transmitting report information to the service providing server 300.

Further, the service providing server 300 receiving the report information determines the regulation violation information of the nearby vehicle based on the black box image or video information or the sensor information included in the report information, and may provide the determined regulation violation information to the vehicle control apparatus 200.

Accordingly, the vehicle control apparatus 200 can determine the retaliatory driving by the vehicle operation of the driver and can perform the vehicle restricting and warning process corresponding thereto and also can check whether a regulation violation corresponding to retaliatory driving of another person by a nearby vehicle has occurred and it also becomes possible to perform a report process.

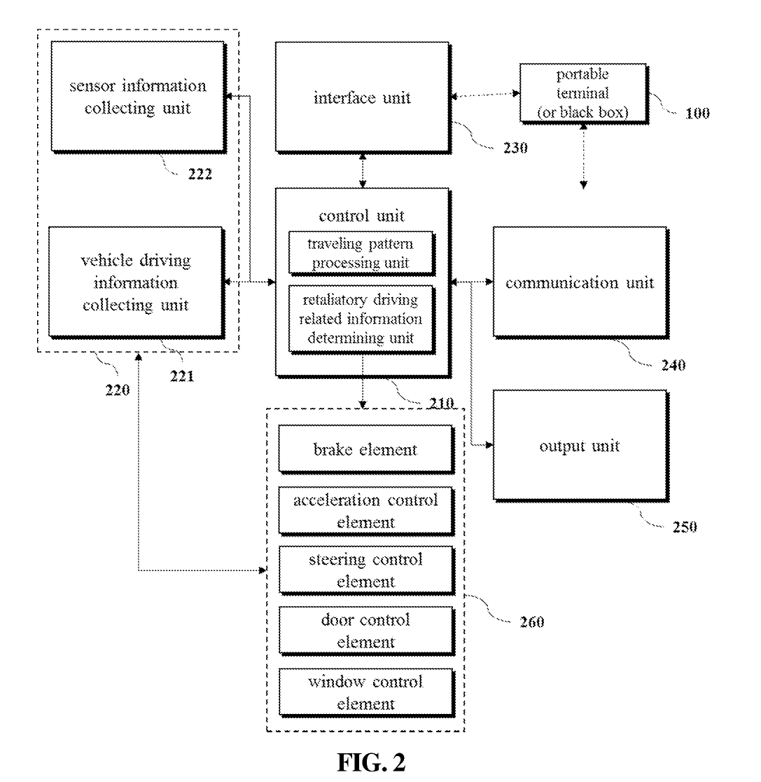

FIG. 2 is a block diagram showing a vehicle control apparatus according to an embodiment of the present invention in more detail.

Referring to FIG. 2, the vehicle control apparatus 200 according to the embodiment of the present invention includes a control unit 210, a driving information collecting unit 220, an interface unit 230, a communication unit 240, an output unit 250, and an electric element unit 260.

First, the control unit 210 may control a plurality of electric elements of the electric element unit 260 provided for vehicle operation according to a user operation input through one or more vehicle control means.

In addition, when the vehicle driving information or the sensor information of the driver is collected from the driving information collecting unit 220, the control unit 210 identifies the driving pattern of the driver from the vehicle driving information or the sensor information, and based on the driving pattern, determines retaliatory driving related information corresponding to the driver, and may include one or more processors for performing a process corresponding to the retaliatory driving related information.

In addition, the control unit 210 may further include a driving pattern processing unit for performing the driving pattern identifying process and a retaliatory driving related information determining unit for determining the retaliatory driving related information.

Also, the plurality of electric elements of the electric element unit 260 may be exemplified by, for example, a brake element, an acceleration control element, a steering control element, a door control element, a window control element and such. However, since this is only an example, various electric parts mounted on a vehicle such as an air conditioning system may be included in the vehicle.

Further, the driving information collecting unit 220 includes a vehicle driving information collecting unit 221 for collecting the vehicle driving information and a sensor information collecting unit 222 for collecting sensor information, and it is possible to transmit the collected vehicle driving information and sensor information to the driving pattern processing unit of the control unit 210.

The vehicle drive information collecting unit 221 may monitor the operation of each electric element unit 260 of the vehicle and may collect the driving information of at least one of a brake element, an acceleration control element, a steering control element, a door control element, and a window control element.

In particular, the vehicle driving information collected by the vehicle driving information collecting unit 221 can be used to determine the retaliatory driving related information. For example, the acceleration and deceleration driving information by the vehicle acceleration control element, the brake element, and the steering element can be processed in the control unit 210.

Further, the sensor information collecting unit 222 collects sensor information sensed by using one or more sensors attached to or installed inside or outside a vehicle, a portable terminal 100, or a driver, and transmits the collected sensor information to the control unit 210.

To this end, the sensor information collecting unit 222 may include various sensor modules. The sensor module includes a body sensing module that senses the driver's body information, a proximity sensing module that senses vehicle information in the vicinity of the driver, a camera sensing module that senses driver's face or motion of a vehicle nearby, or other various sensing modules provided on the portable terminal 100. The sensor information collecting unit 222 may packetize the sensor information collected from the modules to be transmitted to the control unit 210.

Accordingly, the control unit 210 can identify driving pattern information corresponding to the sensor information and the vehicle driving information through the driving pattern processing unit. The control unit 210 can determine retaliatory operation possibility information and type information by comparing the driving pattern information with predetermined retaliatory driving pattern information through the retaliatory driving related information determining unit.

More specifically, the driving pattern processing section can identify the biometrics pattern information from the sensor information for identifying the driving pattern information. The biometric pattern information may be identified from the biometric information of the driver and may include heart rate information sensed by the driver. For example, the sensor information collecting unit 222 may collect heart rate information of a driver from a sensor module attached to or worn by a driver, and may generate and transmit biometric pattern information to the driving pattern processing unit.

Further, the driving pattern processing unit may collect at least one of the acceleration information, the brake information, and the steering information collected from the vehicle driving information collecting unit 221 described above, and accordingly, it is possible to identify the acceleration pattern information for the front, rear, left, and right directions.

Further, the driving pattern processing unit may match the acceleration pattern information with the biometric pattern information to transmit to the retaliatory driving related information determining unit.

Then, the retaliatory driving related information determining unit may determine the retaliatory driving related information including whether there was retaliatory driving of the driver, and types thereof, by matching predetermined retaliatory driving pattern information with the acceleration pattern information during a certain time section and biometric pattern.

Here, the predetermined retaliatory driving pattern information may be accumulated and generated in the service providing server 300 according to learning of the driving pattern information and biometric pattern information for various cases, and the generated pattern information may be provided to the control unit 210 through the communication unit 240.

For example, the retaliatory driving pattern information may include time section pattern information in which the driver's heart rate exceeds 140 or more, and rapid acceleration, deceleration or rapid steering driving pattern information generated after a predetermined time from the time section. When set in such a way, the retaliatory driving related information determining unit can relatively accurately determine the intention of the driver to perform retaliatory driving due to anger or stress caused by another vehicle, and the retaliatory driving related information based thereon can be calculated.

In addition, the control unit 210 can perform a process for preventing retaliatory operation based on the retaliatory driving related information.

First, the control unit 210 may output a related message for each retaliatory driving pattern through an output unit 250 or a portable terminal 100 interlocked therewith, and the related message may include a warning message including information on a penalty provision for each retaliatory driving pattern.

Meanwhile, the control unit 210 may determine that the driver has been subjected to the retaliatory driving of another vehicle according to the retaliatory driving related information, and may perform a process corresponding thereto. In this case, in addition to the retaliatory driving related information, sensing information of vehicles nearby collected by the sensor information collecting unit 222 may be further needed.

For this purpose, the retaliatory driving related information determining unit determines the acceleration, deceleration, and steering information of the nearby vehicle based on the proximity or image information of the vehicle collected from the sensor information collecting unit 222, and based thereon, can determine whether there was any retaliatory driving of nearby vehicles and the type thereof.

For example, the sensor information collecting unit 222 may acquire the black box images or video of the vehicle for a predetermined time associated with the retaliatory driving, and the retaliatory driving-related information determining unit may determine the retaliatory driving-related information based thereon.

In addition, the control unit 210 may process the black box image information into report information according to a user input, and transmit the report information to the service providing server 300 through the communication unit 240. Accordingly, the service providing server 300 can provide report processing and regulation violation information corresponding to the report information, and the control unit 210 can transmit the traffic regulation violation information to the output unit 250 or the portable terminal 100.

Here, the output unit 250 is for generating an output related to a visual, auditory, tactile, etc., and may include a display unit and an audio output module.

In addition, the output unit 250 may be interlocked with a display or an audio output module of the portable terminal 100. The output unit 250 may provide information to be output to the portable terminal 100 through the interface unit 230 or the communication unit 240.

The display unit of the portable terminal 100 or the output unit 250 displays (outputs) information processed by the vehicle control apparatus 200. For example, when the terminal is in the basic mode, a UI (User Interface) or a GUI (Graphic User Interface) related to a call among basic functions can be displayed. Further, when the terminal 100 is a vehicle interface mode providing a vehicle interface, it may display a UI or a GUI associated with the vehicle interface.

Meanwhile, although not shown, the storage unit may store a program for the operation of the control unit 210, and temporarily store input/output data The storage unit may store data for providing vehicle control and interface, and may store important event information and status information associated with the vehicle control and interface.

The storage unit may include at least one storage media selected from the group consisting of a flash memory type, a hard disk type, a multimedia card micro type, a card type storage unit (e.g., an SD or XD storage unit), a RAM (Random Access Memory), SRAM (Static Random Access Memory), ROM (Read-Only Memory), EEPROM (Electrically Erasable Programmable Read-Only Memory), PROM (Programmable Read-Only Memory), a magnetic storage unit, a magnetic disk, and an optical disk.

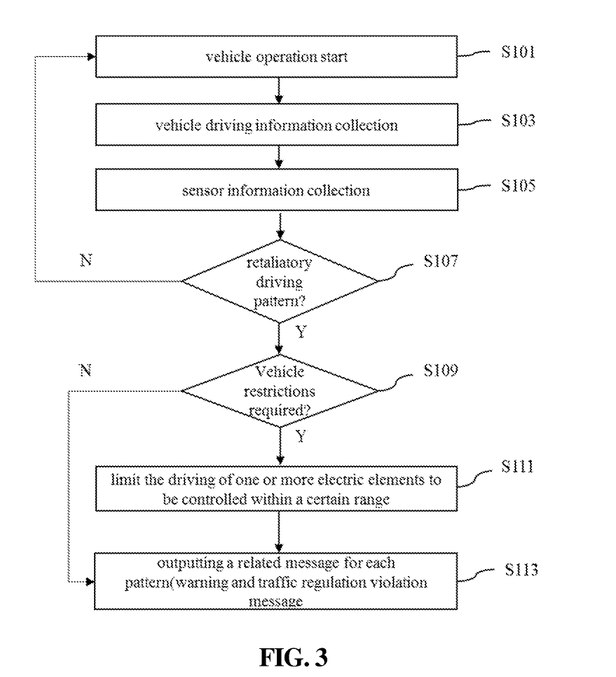

FIG. 3 is a flowchart illustrating an operation of the vehicle control apparatus according to the embodiment of the present invention in more detail.

Referring to FIG. 3, when the operation of the vehicle is started (S101), the vehicle control apparatus 200 according to the embodiment of the present invention collects the vehicle driving information through the vehicle driving information collection unit 221 (S103).

Then, the vehicle control apparatus 200 collects the sensor information through the sensor information collection unit 222 (S105).

In the present embodiment, the sensor information may include biometric pattern information of the driver as described above, and the biometric pattern information may include the heart rate pattern information for each time of the driver.

The vehicle control apparatus 200 determines whether a retaliatory driving pattern is generated based on the vehicle driving information and the sensor information through the retaliatory driving related information determination unit of the control unit 210 (S107).

The retaliatory driving related information determination unit can identify the retaliatory driving possibility based on a comparison with the predetermined retaliatory driving pattern information. Further, the retaliatory driving related information determination unit may determine that the retaliatory driving pattern has occurred when the retaliatory driving possibility is equal to or greater than a predetermined value, and output the retaliatory driving related information according to the generated retaliatory driving pattern to the control unit 210.

For example, the predetermined retaliatory driving pattern information may include a retaliatory driving acceleration pattern information corresponding to the vehicle driving information generated within a predetermined time from a time when the heart rate of the biometric pattern information is equal to or greater than the threshold value. The retaliatory driving acceleration pattern information may include a pattern information wherein acceleration or deceleration exceeding a threshold within a predetermined time is repeated a predetermined number of times or more, in response to the vehicle acceleration information included in the vehicle driving information.

Moreover, the control unit 210 determines whether the vehicle control restriction is required according to the retaliatory driving-related information (S109).

For example, the control unit 210 may set the intensity of the retaliatory driving pattern that needs to be restricted when the retaliatory driving pattern is generated, or may set the retaliatory driving pattern to output only the warning message without limitation. This may be differently set depending on the vehicle, and the control unit 210 may apply the vehicle restriction necessity differently depending on the information of the retaliatory driving pattern.

If a restriction is necessary, the control unit 210 may limit the driving of one or more electric elements of the electric element unit 260 to be controlled within a certain range (S111).

Further, even if the restriction is not applied, the control unit 210 may generate an association message including a warning for each retaliatory driving pattern and a traffic regulation violation message, and output the related message to the output unit 250 or the portable terminal 100 (S113).

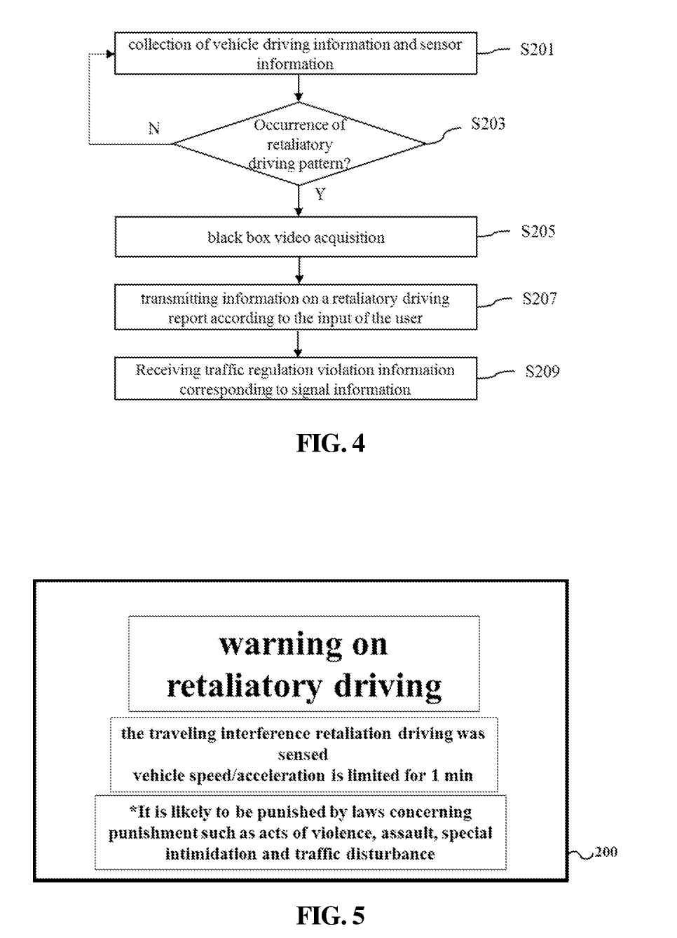

FIG. 4 is a flowchart illustrating an operation of the vehicle control apparatus according to another embodiment of the present invention.

Referring to FIG. 4, the vehicle control apparatus 200 according to another embodiment of the present invention collects vehicle driving information and sensor information through the driving information collecting unit 220 (S201).

Here, the vehicle driving information and the sensor information may further include sensor information of the nearby vehicle.

Accordingly, the control unit 210 determines whether the retaliatory driving pattern information due to retaliatory driving of the nearby vehicle is generated through the retaliatory driving related information determination unit (S203).

As described above, the retaliatory driving related information determining unit can determine the possibility of retaliatory driving damage due to the nearby vehicles by using the image information and the proximity sensing information of the nearby vehicles.

For example, the retaliatory driving related information determination unit may determine the driver's abnormal driving pattern information according to the vehicle driving information and the driver's biometric pattern information, and determine whether the retaliatory driving pattern information of the nearby vehicles corresponding to the abnormal driving pattern information is generated. Thereby, it can be determined whether or not to receive the retaliatory driving damage due to the nearby vehicles.

Accordingly, when the retaliatory driving pattern of the nearby vehicles occurs, the control unit 210 can acquire the black box image information corresponding to the retaliatory driving collected from the inside or outside of the vehicle or the portable terminal 100 through the sensor information collecting unit 222.

Accordingly, the control unit 210 may output the retaliatory driving report information based on the black box image or video information through the output unit 250, and may transmit the retaliatory driving report information through the communication unit 240 according to the user input to the server 300 (S207).

Thereafter, the control unit 210 receives the violation information corresponding to the report information from the service providing server 300 (S209), and outputs the violation information through the portable terminal 100 or the output unit 250.

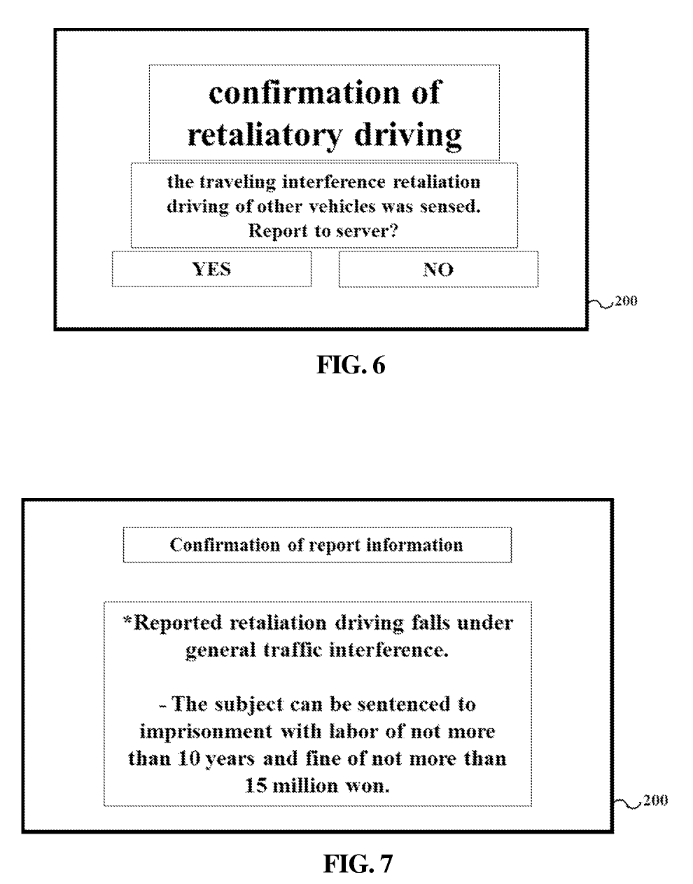

FIGS. 5 to 7 are views showing interface screens of the vehicle control apparatus according to the embodiment of the present invention.

The vehicle control apparatus 200 according to an embodiment of the present invention can output a related message according to the occurrence of the retaliatory driving described above on the interface through the display of the output unit 250 or the portable terminal 100 in various ways.

FIG. 5 shows a retaliatory driving warning and restricting message according to an embodiment of the present invention. As shown in FIG. 5, when the retaliatory driving occurs, the control unit 210 may output the retaliatory driving type information and restriction information of the electrical component unit 60 corresponding thereto. For example, the control unit 210 may perform processing for limiting the driving speed, the acceleration, or the steering angle within a certain range for a predetermined time.

Further, during the processing of the restriction, the control unit 210 can output the punishment provision information together with the above warning message together. This makes it difficult for the driver to perform the retaliatory driving, thereby preventing the retaliatory driving in advance and providing a warning message therefor.

FIGS. 6 and 7 show interface screens in the case where the retaliatory driving of another vehicle is confirmed. First, as shown in FIG. 6, the control unit 210 senses the running interference retaliation driving of another vehicle, and provides an interface for confirming whether or not to report this to the output unit 250 or the portable terminal 100.

Further, as shown in FIG. 7, when the user selects the report, the report information can be transmitted to the service providing server 300. The service providing server 300 can provide the report process and the related traffic regulations information corresponding thereto to the vehicle control apparatus 200.

In FIG. 7, it is exemplified that the retaliatory driving of the nearby vehicles corresponds to general traffic interference, and corresponding penalty provisions can be provided together.

According to the embodiment of the present invention as described above, the driving pattern of the driver is identified from the vehicle (living information and the sensor information collected from the vehicle or the driver, and the retaliatory driving related information corresponding to the driver is determined according to the driving pattern. Thereby, at the time of occurrence of retaliatory driving, this is confirmed and the vehicle driving is physically controlled, thereby preventing the retaliatory driving in advance. Alternatively, the retaliatory driving related information for preventing the retaliatory driving is provided to the user, thereby easily preventing the retaliatory driving and providing the related information.

The methods according to various embodiments of the invention described above may be implemental in program code and provided to each server or device in a state stored in various non-transitory computer readable media.

A non-transitory readable medium is not a medium for storing data for a short time such as a register, a cache, a memory, etc., but means a medium that semi-permanently stores data and can be read by a device. In particular, the various applications or programs described above may be stored and provided on non-volatile readable media such as CD, DVD, hard disk, Blu-ray disk, USB, memory card, ROM, and the like.

Although the preferred embodiments of the present invention have been illustrated and described above, the present invention is not limited to the specific embodiments described above, and various modifications may be made without departing from the spirit of the present invention as claimed in the appended claims. Various modifications can be made by those having ordinary knowledge in the technical field to which the invention belongs, and such modifications will of course not be understood individually from the technical idea and perspective of the present invention.

What is claimed is:

1. A method of controlling a vehicle, the method comprising:
collecting vehicle driving information or sensor information;
identifying a traveling pattern of a driver from the driving information and/or the sensor information;
determining information on a retaliatory driving corresponding to the driver based on the traveling pattern; and
executing a processing corresponding to the information on the retaliatory driving.

2. The method of claim 1, wherein the determining of the information on the retaliatory driving comprises:
determining a possibility of retaliatory driving by comparing the traveling pattern with information on a predetermined retaliatory driving pattern.

3. The method of claim 2, wherein the information on the predetermined retaliatory driving pattern comprises:
information on a retaliatory driving acceleration pattern corresponding to the vehicle driving information, and
wherein the information on the retaliatory driving acceleration pattern comprises pattern information of a pattern of at least a predetermined number of repeats of an acceleration or a deceleration equal to or more than a threshold value within a predetermined time, corresponding to information on a vehicle acceleration included in the vehicle driving information.

4. The method of claim 3, wherein the information on the predetermined retaliatory driving pattern comprises:
information on a retaliatory driving biometric pattern corresponding to the sensor information,
wherein the information on the retaliatory driving biometric pattern comprises information on a heart rate pattern corresponding to heart rate information of the driver included in the sensor information, and
wherein the information on the retaliatory driving biometric pattern comprises matching information on whether there is a match between the information on the heart rate pattern and the information on the retaliatory driving acceleration pattern.

5. The method of claim 1, wherein the executing of the processing comprises:
outputting a related message for each retaliatory driving pattern based on the information on the retaliatory driving.

6. The method of claim 5, wherein the related message comprises:
a warning message including information on a penalty provision for each retaliatory driving pattern.

7. The method of claim 1, wherein the executing of the processing comprises:
limiting a vehicle acceleration or a vehicle speed of the vehicle at a drive unit to a predetermined value or less based on the information on the retaliatory driving.

8. The method of claim 1, wherein the executing of the processing comprises:
upon determining that the driver is subject to a retaliatory driving by another vehicle based on the information on the retaliatory driving, obtaining images from a dashboard camera of the vehicle during a certain period of time related to the retaliatory driving; and
transmitting information on the images from the dashboard camera to a server.

9. The method of claim 8, further comprising:
outputting information on a traffic regulation violation received from the server;
receiving an input of a user report corresponding to the information on the traffic regulation violation; and
transmitting information on a retaliatory driving report based on the black box image according to the input of the user report.

10. An apparatus for controlling a vehicle, the apparatus comprising:
a driving information collecting unit configured to collect vehicle driving information or sensor information;
a traveling pattern processing unit configured to identify a traveling pattern of a driver from the vehicle driving information and/or the sensor information; and
a control unit configured to determine information on a retaliatory driving corresponding to the driver based on the traveling pattern, and to execute a processing corresponding to the information on the retaliatory driving.

11. The apparatus of claim 10, wherein the control unit is configured to determine a possibility of retaliatory driving by comparing the traveling pattern with information on a predetermined retaliatory driving pattern.

12. The apparatus of claim 11, wherein the information on the predetermined retaliatory driving pattern comprises:
information on a retaliatory driving acceleration pattern corresponding to the vehicle driving information, and
wherein the information on the retaliatory driving acceleration pattern comprises pattern information of a pattern of at least a predetermined number of repeats of an acceleration or a deceleration equal to or more than a threshold value within a predetermined time, corresponding to information on a vehicle acceleration included in the vehicle driving information.

13. The apparatus of claim 12, wherein the information on the predetermined retaliatory driving pattern comprises:
information on a retaliatory driving biometric pattern corresponding to the sensor information,
wherein the information on the retaliatory driving biometric pattern comprises information on a heart rate pattern corresponding to heart rate information of the driver included in the sensor information, and
wherein the information on the retaliatory driving biometric pattern comprises matching information on whether there is a match between the information on the heart rate pattern and the information on the retaliatory driving acceleration pattern.

14. The apparatus of claim 10, wherein the control unit is configured to output a related message for each retaliatory driving pattern based on the information on the retaliatory driving, and
wherein the related message comprises a warning message including information on a penalty provision for each retaliatory driving pattern.

15. The apparatus of claim 10, wherein the control unit is configured to limit a vehicle acceleration or a vehicle speed of the vehicle at a drive unit to a predetermined value or less based on the information on the retaliatory driving.

16. The apparatus of claim 10, wherein the control unit is configured to
obtain images, upon determining that the driver is subject to a retaliatory driving by another vehicle based on the information on the retaliatory driving, from a dashboard camera of the vehicle during a certain period of time related to the retaliatory driving, and
further comprise a transmission unit configured to transmit information on the images from the dashboard camera to a server.

17. The apparatus of claim 16, wherein the transmission unit further comprises:
a display unit configured to output information on a traffic regulation violation received from the server; and
a receiving unit configured to receive an input of a user report corresponding to the information on the traffic regulation violation, and
wherein the transmission unit configured to transmit information on a retaliatory driving report based on the black box image according to the input of the user report.

18. A non-transitory computer readable medium storing a computer program including computer-executable instructions for causing, when executed in a processor, the processor to perform the method according to claim 1.
